(19) United States
(12) Patent Application Publication
(10) Pub. No.: US 2019/0282644 A1
(43) Pub. Date: Sep. 19, 2019

(54) ANTIDIURETIC FORMULATION

(71) Applicant: Fei SUN, Bruce (AU)

(72) Inventor: Fei SUN, Bruce (AU)

(21) Appl. No.: 16/355,349

(22) Filed: Mar. 15, 2019

(30) Foreign Application Priority Data
Mar. 15, 2018 (AU) 2018900855

(51) Int. Cl. A61K 36/28; A61K 36/57; A61K 36/076; A61K 36/54; A61K 36/884; A61P 13/02

(52) U.S. Cl. CPC A61K 36/28; A61K 36/57; A61P 13/02; A61K 36/54; A61K 36/884; A61K 36/076

(57) ABSTRACT

An exemplary formulation including a specific weight ratio of Atractylodes macrocephala to other certain ingredients surprisingly had an antidiuretic effect which is opposite to what was expected from the common general knowledge. One aspect of such a formulation includes Atractylodes macrocephala; at least one other ingredient that can include at least one of Polyporus umbellatus; Poria cocos; Cinnamomum cassia (twig); and at least one of Alisma orientale and Alisma plantago-aquatica. The formulation can be formed with a weight ratio of Atractylodes macrocephala being greater than 25% with respect to the at least one other ingredient. These formulations may be useful in a method for the treatment of excessive urination, the method including administering an effective amount of a formulation as described herein to a patent in need thereof.

### FIELD OF THE INVENTION

This invention relates generally to antidiuretic formulations.

### BACKGROUND OF THE INVENTION

Bai Zhu (Atractylodes Macrocephala), also known as White Atractylodes Rhizome or Atractylodes Macrocephala Rhizome, refers to the root of Atractylodes macrocephala Koidz. which is a perennial herb typically being 30 to 60 in height.

The rhizome is fleshy and clenched like a first with the stem rising erect therefrom and branching outwardly in the upper part thereof.

The rhizome is usually 3 to 13cm long, and 1.5 to 7cm in diameter. The surface of the rhizome is greyish yellow or greyish brown in colour, with tubercule and intermittent lengthwise wrinkles and fibril scars. The texture is hard and difficult to break. The traverse cross section is uneven, yellowish white to light brown, and scattered with brownish yellow oil spots. The rhizome has a delicate fragrance and sweet but pungent taste and is sticky when chewed.

The rhizome is used for medicine which is collected in winter, cleaned, and dried over a fire or in the sun.

Atractylodes macrocephala has been commonly known and used for centuries to induce diuresis.

For example the original text of Chinese medicine of Shang Han Lun (written around 200 AD) states “When floating pulse, urine negative [i.e. lack of urination], slight fever, thirst, use Wu Ling San.(Alisma orientale 30.5z, Atractylodes macrocephalal8z, Cinnamomum cassia twigl2z, Polyporus umbellatusml8z, Poria cocos18z)”.

Atractylodes macrocephala is also known for adjusting gastrointestinal motility, treating ulcers, protecting the liver, improving the immune system, relieving stress, enhancing hematopoietic function, reducing oxidation, slowing down aging, regulating blood sugar level, treating indigestion, treating stomach-ache, treating bloating, treating fluid retention, treating diarrhea, treating loss of appetite, treating weight loss due to cancer, treating allergies to dust mites, and treating joint pain (rheumatism) amongst others. Indeed, in Shang Han Lun (Treatise on Febrile Diseases) and Jin Gui Yao Lue (Synopsis of Golden Chamber), Atractylodes macrocephala has been enlisted in 35 formulas.

### SUMMARY OF THE DISCLOSURE

We surprisingly observed that a specific weight ratio of Atractylodes macrocephala surprisingly confers antidiuretic effects. An antidiuretic is a substance that reduces urination, opposing diuresis and has the opposite effect of a diuretic.

Specifically, we found that a formulation comprising a specific weight ratio of Atractylodes macrocephala to other certain ingredients surprisingly had a antidiuretic effect which is opposite to what was expected from the common general knowledge. This is puzzling because, as above, Atractylodes macrocephala is traditionally known for its diuretic properties.

As such, there is provided herein a formulation having the provided increased weight ratio of Atractylodes macrocephala that therefore may be used for the treatment of excessive urination.

According to one aspect, there is provided a formulation comprising: Atractylodes macrocephala; at least one other ingredient comprising at least one of: Polyporus umbellatus; Poria cocos; Cinnamomum cassia (twig); and at least one of Alisma orientale and Alisma plantago-aquatica, wherein the formulation comprises a weight ratio of Atractylodes macrocephala being greater than 25% with respect to the at least one other ingredient.

The formulation may comprise a weight ratio of the at least one other ingredient being less than 25% with respect to the at least one other ingredient.

The formulation may comprise a weight ratio of Atractylodes macrocephala being greater or equal to approximately 30% with respect to the at least one other ingredient.

The at least one other ingredient may comprise all of: Polyporus umbellatus; Poria cocos; Cinnamomum cassia (twig); and at least one of Alisma orientale and Alisma plantago-aquatica

The formulation may comprise a ratio of: Atractylodes macrocephala at greater than 250 mg/g; Polyporus umbellatus at less than 250 mg/g; Poria cocos at less than 250 mg/g; Cinnamomum cassia (twig) at less than 250 mg/g; and at least one of Alisma orientale or Alisma plantago-aquatica at less than 250 mg/g.

The formulation may comprise a ratio of: Atractylodes macrocephala at approximately 300 mg/g; Polyporus umbellatus at approximately 100 mg/g; Poria cocos at approximately 200 mg/g; Cinnamomum cassia (twig) at approximately 66 mg/g; and and Alisma orientale or Alisma plantago-aquatica at approximately 100 mg/g.

There is also provided a method for the treatment of excessive urination, the method comprising administering a formulation as claimed in any one of the claims.

Other aspects of the invention are also disclosed.

### DESCRIPTION OF EMBODIMENTS

There is provided herein a formulation of specific weight ratios of ingredients. The ingredients of the formulation are those of the Wu Ling San formula comprising Atractylodes macrocephala, Alisma orientale (or Alisma plantago-aquatica), Polyporus umbellatusm, Poria cocos and Cinnamomum cassia twig.

The effects of these individual ingredients are commonly known as follows:

| Ingredient | Known effect |
|---|---|
| Alisma orientale (ze xie) | Diuretic |
| Polyporus umbellatus (zhu Ling) | Diuretic |
| Poria cocos (Fu Ling) | Diuretic |
| Atractylodes macrocephala (bai zhu) | Diuretic |

We observed that increasing the relative weight ratio of Atractylodes macrocephala to the other ingredients surprisingly had an antidiuretic effect.

In one exemplary formulation described in further detail below, we increased the weight ratio of Atractylodes macrocephala to 39.2% as compared to, for example Polyporus umbellatus at 13.1%, Poria cocos at 26.1%, Cinnamomum cassia at 8.6%, and Alisma orientale, or Alisma plantago-aquatica at 13.1%.

The table below provides various prior art TGA approved formulations 1-5 as compared to the present formulation providing the respective ingredient ratios.

| | | | | | Alisma | | |
|---|---|---|---|---|---|---|---|
| | | | | | orientale, | | |
| | | | | | or | | |
| | | | | | Alisma | | |
| | Polyporus | Poria | Atractylodes | Cinnamomum | plantago- | | |
| | umbellatus | cocos | macrocephala | cassia | aquatica | Glycyrrhiza | Phyllostachys |
| Prior art formulation 1 | 18.8% | 18.8% | 18.8% | 12.5% | 31.2% | 0.0% | 0.0% |
| Sponsor: Great Grand Care Pty Ltd | | | | | | | |
| ARTG Entry: 219956 0700 (kaiser | | | | | | | |
| pharmaceutical) wu ling san | | | | | | | |
| (poria five herb formula) | | | | | | | |
| Therapeutic effect: Disinhibit water, | | | | | | | |
| dispel damp, drain heat, and resolve | | | | | | | |
| the exterior | | | | | | | |
| Prior art formulation 2 | 15.0% | 25.0% | 15.0% | 15.0% | 20.0% | 2.0% | 8.0% |
| Sponsor: Herbal international Pty Ltd | | | | | | | |
| ARTG Entry: 79788 Wu Ling San | | | | | | | |
| Therapeutic effect: Diuretic. Aids or | | | | | | | |
| assists in the treatment of fluid | | | | | | | |
| retention | | | | | | | |
| Prior art formulation 3 | 20.0% | 25.0% | 25.0% | 10.0% | 20.0% | 0.0% | 0.0% |
| Sponsor: Shen neng herbal medicines | | | | | | | |
| ARTG Entry: 76149 Wu Ling San | | | | | | | |
| Therapeutic effect: Aids or assists in | | | | | | | |
| the prevention of muscular cramps | | | | | | | |
| and spasms | | | | | | | |
| Prior art formulation 4 | 22.7% | 22.7% | 22.7% | 13.7% | 18.2% | 0.0% | 0.0% |
| Sponsor: Sun herbal Pty Ltd | | | | | | | |
| ARTG Entry: 120745 wu ling san | | | | | | | |
| a.k.a. hoelen five formula | | | | | | | |
| Therapeutic effect: Diuretic. Aids or | | | | | | | |
| assists in the treatment of fluid | | | | | | | |
| retention. aids digestion. | | | | | | | |
| Prior art formulation 5 | 20.0% | 26.7% | 20.0% | 13.3% | 20.0% | 0.0% | 0.0% |
| Sponsor: Cathay herbal laboratories | | | | | | | |
| ARTG Entry: 41843 wu ling san | | | | | | | |
| (hoelen five formula) | | | | | | | |
| Therapeutic effect: traditionally used | | | | | | | |
| to clear dampness and help the | | | | | | | |
| fluid metabolism | | | | | | | |
| Present formulation | 13.1% | 26.1% | 39.2% | 8.6% | 13.1% | 0.0% | 0.0% |
| Therapeutic effect: reduce excess | | | | | | | |
| urination | | | | | | | |

As can be seen from the above, Atractylodes macrocephala predominates in the present formulation as compared to prior art formulations. Specifically, Atractylodes macrocephala is provided in the ratio of greater than 25% with respect to any one of Polyporus umbellatus, Poria cocos, Cinnamomum cassia, and Alisma orientale, or Alisma plantago-aquatica.

As is also apparent from the above table, the other prior art formulations 1-5 provide and are registered in respect of therapeutic effects other than for antidiuretic effect.

It should be noted that the specific ratios of the present formulation may be varied provided Atractylodes macrocephala predominates by weight ratio by more than approximately 25%. For example, the present formulation could be provided according to the following ratios: Atractylodes macrocephala at 300 mg/g, Polyporus umbellatus at 100 mg/g, Poria cocos at 200 mg/g, Cinnamomum cassia (twig) at 66 mg/g, and Alisma orientale or Alisma plantago-aquatica at 100 mg/g.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

1. A formulation comprising:
Atractylodes macrocephala;
at least one other ingredient comprising at least one of:
Polyporus umbellatus;
Poria cocos;
Cinnamomum cassia (twig); and
at least one of Alisma orientale and Alisma plantago-aquatica, wherein the formulation comprises a weight ratio of Atractylodes macrocephala being greater than 25% with respect to the at least one other ingredient.

2. A formulation as claimed in claim 1, wherein the formulation comprises a weight ratio of the at least one other ingredient being less than 25% with respect to the at least one other ingredient.

3. A formulation as claimed in claim 1, wherein the formulation comprises a weight ratio of Atractylodes macrocephala being greater or equal to approximately 30% with respect to the at least one other ingredient.

4. A formulation as claimed in claim 1, wherein the at least one other ingredient comprises all of:
Polyporus umbellatus;
Poria cocos;
Cinnamomum cassia (twig); and
at least one of Alisma orientale and Alisma plantago-aquatica

5. A formulation as claimed in claim 4, wherein the formulation comprises a ratio of:
Atractylodes macrocephala at greater than 250 mg/g;
Polyporus umbellatus at less than 250 mg/g;
Poria cocos at less than 250 mg/g;
Cinnamomum cassia (twig) at less than 250 mg/g; and
at least one of Alisma orientale or Alisma plantago-aquatica at less than 250 mg/g.

6. A formulation as claimed in claim 4, the formulation comprises ratio of:
Atractylodes macrocephala at approximately 300 mg/g;
Polyporus umbellatus at approximately 100 mg/g;
Poria cocos at approximately 200 mg/g;
Cinnamomum cassia (twig) at approximately 66 mg/g; and
and Alisma orientale or Alisma plantago-aquatica at approximately 100 mg/g.

7. A method for the treatment of excessive urination, the method comprising administering a formulation as claimed in claim 1.

8. A method for the treatment of excessive urination, the method comprising administering a formulation as claimed in claim 2.

9. A method for the treatment of excessive urination, the method comprising administering a formulation as claimed in claim 3.

10. A method for the treatment of excessive urination, the method comprising administering a formulation as claimed in claim 4.

11. A method for the treatment of excessive urination, the method comprising administering a formulation as claimed in claim 5.

12. A method for the treatment of excessive urination, the method comprising administering a formulation as claimed in claim 6.
